(19) United States
(12) Patent Application Publication
(10) Pub. No.: US 2026/0026720 A1
(43) Pub. Date: Jan. 29, 2026

(54) IMPLANTABLE MEDICAL DEVICE CONFIGURED FOR OPTICAL SENSING

(71) Applicant: Medtronic, Inc., Minneapolis, MN (US)

(72) Inventors: Nicholas H. Finstrom, Coon Rapids, MN (US); Aaron B. Bartnik, Osseo, MN (US); Mark R. Boone, Gilbert, AZ (US); Patrick M. Hermans, Lake Elmo, MN (US)

(21) Appl. No.: 19/277,772

(22) Filed: Jul. 23, 2025

Related U.S. Application Data
(60) Provisional application No. 63/675,941, filed on Jul. 26, 2024.

(51) Int. Cl. A61B 5/1455; A61B 5/0215; A61B 5/026; A61B 5/287

(52) U.S. Cl. CPC A61B 5/14552; A61B 5/02154; A61B 5/0261; A61B 5/287; A61B 2562/0242

(57) ABSTRACT

An example implantable medical device includes a housing comprising an optical window; an optical sensor within the housing and configured to emit light to the optical window; and a masking on a portion of the optical window, the masking configured to absorb a portion of the emitted light internally reflected within the optical window.

### RELATED APPLICATIONS

This application claims the benefit of U.S. Provisional Application Ser. No. 63/675,941, filed Jul. 26, 2024 the entire contents of each of which are incorporated herein by reference.

### TECHNICAL FIELD

This disclosure generally relates to sensing patient parameters, and more particularly optically sensing, via an implantable medical device.

### BACKGROUND

Various implantable medical devices (IMDs) have been clinically implanted or proposed for therapeutically treating or monitoring one or more conditions of a patient. Such devices may be adapted to monitor or treat conditions or functions relating to heart, muscle, nerve, brain, stomach, endocrine organs or other organs and their related functions. Advances in design and manufacture of miniaturized electronic and sensing devices have enabled development of implantable devices capable of therapeutic as well as diagnostic functions such as pacemakers, cardioverters, defibrillators, biochemical sensors, implantable loop recorders, and pressure sensors, among others. Such devices may be associated with leads that position electrodes or sensors at a desired location or may be leadless with electrodes and/or sensors integrated into the device housing. These devices may have the ability to wirelessly transmit data either to another device implanted in the patient or to another instrument located externally of the patient, or both.

### SUMMARY

In general, this disclosure is directed to systems, devices, and techniques for measuring and/or determining a physiological characteristic of a patient based on an optical signal of an optical sensor. In particular, methods and devices disclosed herein are directed to attenuating light emitted by a light source of an optical sensor that is reflected internally in an IMD, attenuating ambient light from a detector of an optical sensor, and/or reducing reflection and/or refraction of light emitted by a light source of an optical sensor at optical interfaces into and out of an IMD. Masking and/or optical design may attenuate light reflected internally in an IMD, attenuate ambient light incident on an optical detector, and/or reduce reflection and/or refraction of light emitted by a light source of an optical sensor may improve the percentage of optical sensing light (e.g., light that is emitted by a light source of an optical sensor that passes into tissue and interacts with tissue, such as through absorption and scattering) that is detected by a detector of the optical sensor. In some examples, improving the percentage of optical sensing light that is detected by a detector of the optical sensor may improve the accuracy of a physiological characteristic (e.g., a physiological signal or parameter) determined based on light detected by detector.

In one example, this disclosure describes an implantable medical device comprising: a housing comprising an optical window; an optical sensor within the housing and configured to emit light to the optical window; and a masking on a portion of the optical window, the masking configured to absorb a portion of the emitted light internally reflected within the optical window.

In another example, this disclosure describes a method comprising: emitting light, by a light source of an optical sensor positioned in an implantable medical device (IMD), to an optical window of the IMD and towards a tissue of the patient, the light including optical sensing light that is to interact with the tissue of the patient and reflected light that is to be reflected in the IMD; blocking, by masking positioned on a portion of the optical window, at least a portion of the reflected light in the IMD; detecting, by a detector, the optical sensing light; and determining, by processing circuitry and based, at least, on the detected optical sensing light, the physiological characteristic of the patient.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below. Other features, objects, and advantages will be apparent from the description, drawings, and claims.

### BRIEF DESCRIPTION OF DRAWINGS

FIG. 1 is a conceptual drawing illustrating an example medical system in conjunction with a patient, according to various examples described in this disclosure.

FIG. 2 is a functional schematic diagram of an example implantable medical device (IMD), according to various examples described in this disclosure.

FIG. 3 is a conceptual perspective schematic diagram of the example IMD of FIG. 2, according to various examples described in this disclosure.

FIG. 4 is a block diagram illustrating the example IMD of FIGS. 1-3.

FIG. 5 is an example of a conceptual cross-sectional side-view of the example IMD of FIGS. 1-4 including masking, according to various examples described in this disclosure.

FIG. 6 is an example of a conceptual cross-sectional side-view of the example IMD of FIGS. 1-4 including masking and an integrated lens mask, according to various examples described in this disclosure.

FIG. 7 is an example of a conceptual cross-sectional side-view of the example IMD of FIGS. 1-4 including masking and underfill, according to various examples described in this disclosure.

FIG. 8 is a conceptual cross-sectional side-view of an example of an integrated lens of an optical window, according to various examples described in this disclosure.

FIG. 9 is a flow diagram of an example method of determining a physiological characteristic of a patient, according to various examples described in this disclosure.

In the figures, use of a same reference number or a same reference number with a letter extension may be used to indicate a same or corresponding device or element when used in a same drawing or in different drawings. In addition, unless otherwise indicated, devices and/or other objects such as a patient, an implantable medical device, or an electrical device such as an electrical coil, are not necessarily illustrated to scale relative to each other and/or relative to an actual example of the item being illustrated. In particular, various drawings provided with this disclosure illustrate a “patient” represented by a human-shaped outline and are not to be considered drawn to scale relative to an actual human patient or with respect to other objects illustrated in the same figure unless otherwise specifically indicated in the figure for example by dimensional indicators, or for example as otherwise described in the text of the disclosure.

### DETAILED DESCRIPTION

A variety of types of medical devices sense physiological signals or parameters of a patient. An implantable medical device (IMD) may include an optical sensor configured to sense optical signals from which processing circuitry of a system including the IMD may determine physiological signal values or parameter values. The optical sensor may be integrated with a housing of the IMD. Example IMDs that may be configured to sense optical signals that may be used to monitor physiological signals or parameters include pacemakers and implantable cardioverter-defibrillators, which may be coupled to intravascular or extravascular leads, as well as pacemakers with housings configured for implantation within the patient, which may be leadless. An example of pacemaker configured for intracardiac implantation is the Micra™ Transcatheter Pacing System, available from Medtronic, Inc. Some IMDs that do not provide therapy, e.g., implantable patient monitors, may be configured to sense optical signals that may be used to monitor physiological signals or parameters. One example of such an IMD is the Reveal LINQ™ and LINQ II™ Insertable Cardiac Monitors (ICMs), available from Medtronic, Inc., which may be inserted subcutaneously. Such IMDs may facilitate relatively longer-term monitoring of patients during normal daily activities, and may periodically transmit collected data to a network service, such as the Medtronic Carelink™ Network.

In some examples of the techniques described herein, an IMD including an optical sensor may include one or more of masking, integrated lens, underfill, and/or backfill, positioned at particular locations of the IMD, such as with respect to optical emitters, detectors and/or windows to allow light transport into and out of the body to reduce internally reflected light and/or ambient light received by a detector of an optical sensor to improve the percentage of optical sensing light (e.g., light that is emitted by a light source of the optical sensor and passes into tissue and interacts with tissue, such as through absorption and scattering) that is detected by the detector of the optical sensor.

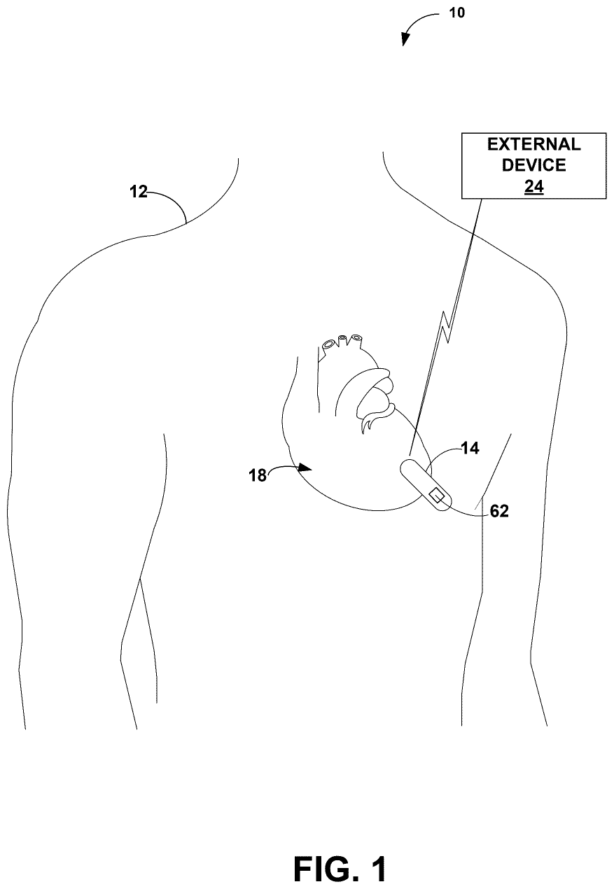

FIG. 1 is a conceptual drawing illustrating an example medical system 10 in conjunction with a patient 12, according to various examples described in this disclosure. The systems, devices, and methods described in this disclosure may include example optical sensors included with IMD 14, as illustrated and described with respect to FIG. 1. For purposes of this description, knowledge of cardiovascular anatomy and functionality is presumed, and details are omitted except to the extent necessary or desirable to explain the context of the techniques of this disclosure. System 10 includes IMD 14 including optical sensor 62, implanted at or near the site of a heart 18 of a patient 12 and an external computing device 24.

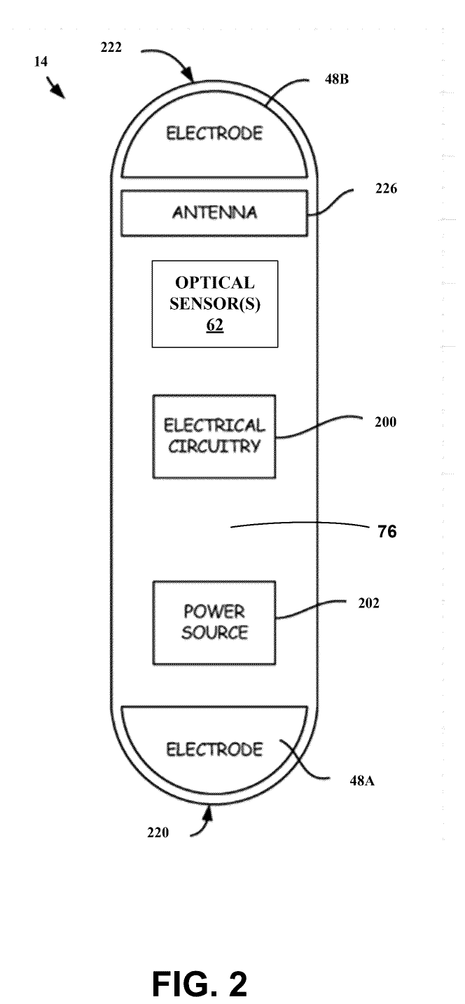

Example techniques disclosed herein may be used with IMD 14, which may be in wireless communication with at least one of external device 24 and other devices not pictured in FIG. 1. In some examples, IMD 14 is implanted outside of a thoracic cavity of patient 12 (e.g., subcutaneously in the pectoral location illustrated in FIG. 1). IMD 14 may be positioned near the sternum near or just below the level of the heart of patient 12, e.g., at least partially within the cardiac silhouette. IMD 14 includes a plurality of electrodes 48 (FIGS. 2-7) and is configured to sense a cardiac electrogram (EGM) via the plurality of electrodes, as well as other physiological signals and/or parameters via optical sensor 62. In some examples, IMD 14 takes the form of the LINQ™ or LINQ II™ ICM, or another ICM similar to, e.g., a version or modification of, the LINQ™ or LINQ II™ ICM. Although described primarily in the context of examples in which IMD 14 is an ICM, in various examples, IMD 14 may represent a cardiac monitor, a defibrillator, a cardiac resynchronization pacer/defibrillator, a pacemaker, an implantable pressure sensor, a neurostimulator, or any other implantable or external medical device.

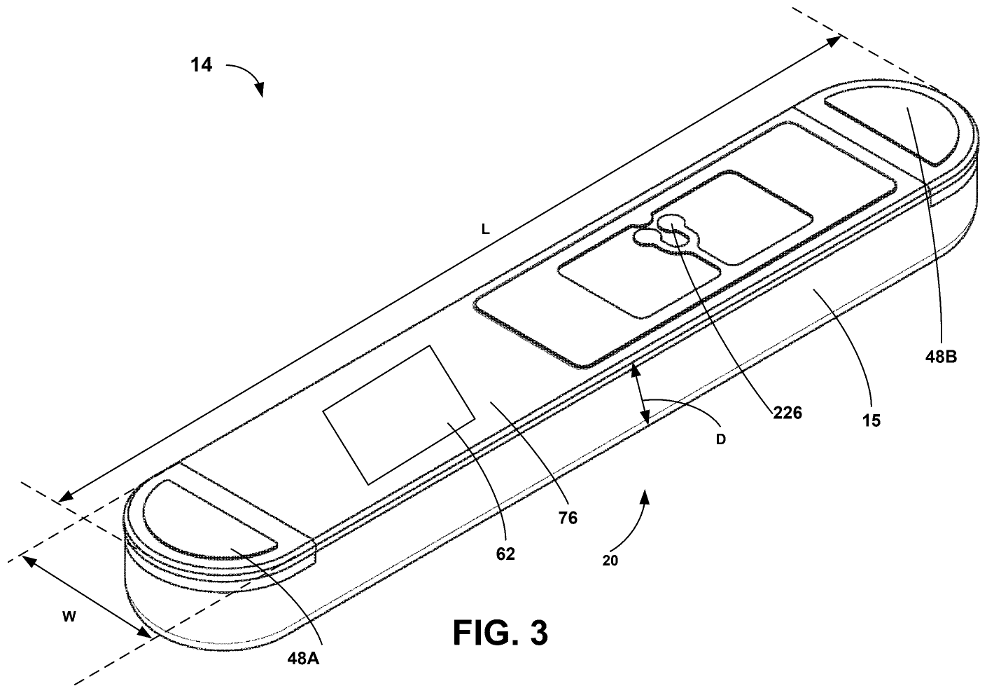

In some examples, IMD 14 is defined by a length L, a width W and thickness or depth D and is in the form of an elongated rectangular prism wherein the length L is much larger than the width W, which in turn is larger than the depth D, as illustrated in FIG. 3 below. In one example, the geometry of the IMD 14—in particular a width W greater than the depth D—is selected to allow IMD 14 to be inserted under the skin of the patient using a minimally invasive procedure and to remain in the desired orientation during insert. For example, IMD 14 may include a radial asymmetry (notably, a rectangular shape) along the longitudinal axis that maintains the device in the proper orientation following insertion. For example, in one example the spacing between electrode 48A and electrode 48B may range from 30 millimeters (mm) to 55 mm, 35 mm to 55 mm, and from 40 mm to 55 mm and may be any range or individual spacing from 25 mm to 60 mm. In another example the spacing between electrode 48A and electrode 48B may range from 15 mm to 30 mm, 17 mm to 28 mm, and from 20 mm to 28 mm and may be any range or individual spacing from 12 mm to 30 mm. In addition, IMD 14 may have a length L that ranges from 30 mm to about 70 mm. In other embodiments, the length L may range from 40 mm to 60 mm, 45 mm to 60 mm and may be any length or range of lengths between about 30 mm and about 70 mm. In some examples, IMD 14 may have a length L that ranges from 15 mm to about 35 mm, or from 20 mm to 30 mm, 22 mm to 30 mm and may be any length or range of lengths between about 15 mm and about 35 mm. In addition, the width W of a major surface of IMD 14, e.g., insulative cover 76 in the example shown, may range from 3 mm to 10 mm and may be any single or range of widths between 3 mm and 10 mm, or may range from 1.5 mm to 5 mm and may be any single or range of width between 1.5 mm and 5 mm. The thickness of depth D of IMD 14 may range from 2 mm to 9 mm, or from 1.5 mm to 4.5 mm. In other embodiments, the depth D of IMD 14 may range from 2 mm to 5 mm and may be any single or range of depths from 2 mm to 9 mm, or may range from 1 mm to 2.5 mm and may be any single or range of depts from 1 mm to 4.5 mm. In addition, IMD 14 according to an example of the present invention has a geometry and size designed for ease of implant and patient comfort. Examples of IMD 14 described in this disclosure may have a volume of 3 cubic centimeters (cm) or less, 1.5 cubic cm or less or any volume between 3 and 1.5 cubic centimeters, or may have a volume of 1.5 cubic centimeters (cm) or less, 0.75 cubic cm or less or any volume between 1.5 and 0.75 cubic centimeters.

External device 24 may be a computing device with a display viewable by the user and an interface for providing input to external device 24 (i.e., a user input mechanism). In some examples, external device 24 may be a notebook computer, tablet computer, workstation, one or more servers, cellular phone, personal digital assistant, or another computing device that may run an application that enables the computing device to interact with IMD 14. External device 24 is configured to communicate with IMD 14 and, optionally, another computing device (not illustrated in FIG. 1), via wireless communication. External device 24, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

External device 24 may be used to configure operational parameters for IMD 14. External device 24 may be used to retrieve data from IMD 14. The retrieved data may include values of physiological parameters measured by IMD 14, indications of episodes of arrhythmia or other maladies detected by IMD 14, and physiological signals recorded by IMD 14, e.g., from optical sensor 62. For example, external device 24 may retrieve cardiac EGM segments recorded by IMD 14, e.g., due to IMD 14 determining that an episode of arrhythmia or another malady occurred during the segment, or in response to a request to record the segment from patient 12 or another user. In some examples, one or more remote computing devices may interact with IMD 14 in a manner similar to external device 24, e.g., to program IMD 14 and/or retrieve data from IMD 14, via a network.

In some examples, external device 24 may include one or more light sources that transmit light into the tissue creating a transmission mode sensor rather than reflection mode sensor (e.g., light source is in IMD 14). In some examples, both IMD 14 and external device 24 may have one or more light sources and may measure physiological characteristics of a plurality of different tissue volumes based on location of external device 24 and emitter selection on external device 24 or IMD 14. In some examples, external device 24 may block ambient light (e.g., light from a different light source external to optical sensor) that may interfere with measurements of light detected by optical sensor 62. In some examples, reflected light 408 and/or ambient light may contribute to noise or reduce the effective dynamic range of detector 64 and external device 24 may increase the received light signal to noise ratio by reducing noise factors.

In various examples, IMD 14 may include one or more additional sensor circuits configured to sense a particular physiological or neurological parameter associated with patient 12, or may comprise a plurality of sensor circuits, which may be located at various and/or different positions relative to patient 12 and/or relative to each other and may be configured to sense one or more physiological parameters associated with patient 12.

For example, IMD 14 may include a sensor operable to sense a body temperature of patient 12 in a location of the IMD 14, or at the location of the patient where a temperature sensor coupled by a lead to IMD 14 is located. In another example, IMD 14 may include a sensor configured to sense motion, such as steps taken by patient 12 and/or a position or a change of posture of patient 12. In various examples, IMD 14 may include a sensor that is configured to detect breaths taken by patient 12. In various examples, IMD 14 may include a sensor configured to detect heartbeats or heart rhythm of patient 12.

In various examples, IMD 14 includes optical sensor 62 that is configured to measure one or more physiological parameters of patient 12, such as systemic blood pressure of patient 12, an oxygenation of blood of patient 12, and/or blood movement within tissue and/or vasculature of patient 12 which may be indicative of pulse pressure waveforms. In some examples, e.g., as illustrated in FIGS. 3 and 5-7, IMD 14 may include a housing 20 comprising an optical window 75 and an optical sensor 62 within housing 20. In some examples, the optical sensor 62 of IMD 14 includes one or more light sources 63 configured to emit light 406 to the optical window 75 towards tissue of patient 12, e.g., a photo emitter such as a light emitting diode (LED), a laser such as a vertical cavity surface emitting laser (VCSEL), or any suitable light source. Optical sensor 62 also includes one or more detectors 64 configured to detect the light emitted by the light source after the light interacts with the tissue 402 of the patient.

Processing circuitry of IMD 14 or external device 24 may be configured to determine a physiological characteristic (e.g., a physiological signal or parameter) of patient 12 based on optical sensing light (e.g., light that is emitted by a light source 63 of optical sensor 62 and passes into tissue 402 and interacts with tissue 402, such as through absorption and scattering) detected by one or more detector(s) 64 of optical sensor 62. In some examples, reflected light 408 (e.g., light that is from one or more of light source 63 of optical sensor 62 that is reflected internally within IMD 14) and/or ambient light (e.g., light from a different light source external to optical sensor 62) from within tissue 402 may interfere with measurements of light detected by optical sensor 62. In some examples, reflected light 408 and/or ambient light may contribute to noise or reduce the effective dynamic range of detector 64.

In some examples of the techniques described herein, IMD 14 may include masking 66, integrated lens 67, underfill 68, and/or backfill 69, such as described herein, to reduce reflected light 408 and/or ambient light received by detector 64, which improves the percentage of optical sensing light (e.g., light that is emitted by a light source 63 of optical sensor 62 and passes into tissue 402 and interacts with tissue 402, such as through absorption and scattering) that is detected by detector 64. In some examples, improving the percentage of optical sensing light that is detected by detector 64 may improve the accuracy of a physiological characteristic (e.g., a physiological signal or parameter) determined by IMD 14 or external device 24 based on light detected by detector 64.

In some examples, one or more of the sensors comprising IMD 14 may be implanted within patient 12, that is, implanted below at least the skin level of the patient. In various examples, IMD 14 may be configured to sense one or more physiological parameters associated with patient 12, and to transmit data corresponding to the sensed physiological parameter or parameters to the external device 24, as represented by the lightning bolt coupling IMD 14 to the external device 24.

Transmission of data from IMD 14 to external device 24 in various examples may be performed via wireless transmission, using for example any of the formats for wireless communication described above. In various examples, IMD 14 may communicate wirelessly to an external device (e.g., an instrument or instruments) other than or in addition to external device 24, such as a transceiver or an access point that provides a wireless communication link between IMD 14 and a network. Examples of communication techniques used by any of the devices described above with respect to FIG. 1 may include radiofrequency (RF) telemetry, which may be an RF link established via Bluetooth®, Wi-Fi, Near Field Communication (NFC), or medical implant communication service (MICS).

In some examples, system 10 may include more or fewer components than depicted in FIG. 1. For example, in some examples, system 10 may include multiple additional IMDs, such as implantable pacemaker devices or other IMDs, implanted within patient 12. In these examples, IMD 14 may function as a hub device for the other IMDs. For example, the additional IMDs may be configured to communicate with the IMD 14, which would then communicate to the external device 24, such as a user's smartphone, via a low-energy telemetry protocol. IMD 14 may provide a theoretically infinite energy capacity, in that IMD 14 may not need to be replaced or otherwise removed. Accordingly, IMD 14 may provide the ability to more-frequently telemeter information, as well as more-active titration of therapies.

For the remainder of the disclosure, a general reference to a medical device system may refer collectively to include any examples of medical device system 10, a general reference to IMD 14 may refer collectively to include any examples of IMD 14, a general reference to sensor circuits may refer collectively to include any examples of sensor circuits of IMD 14, and a general reference to an external device may refer collectively to any examples of external device 24.

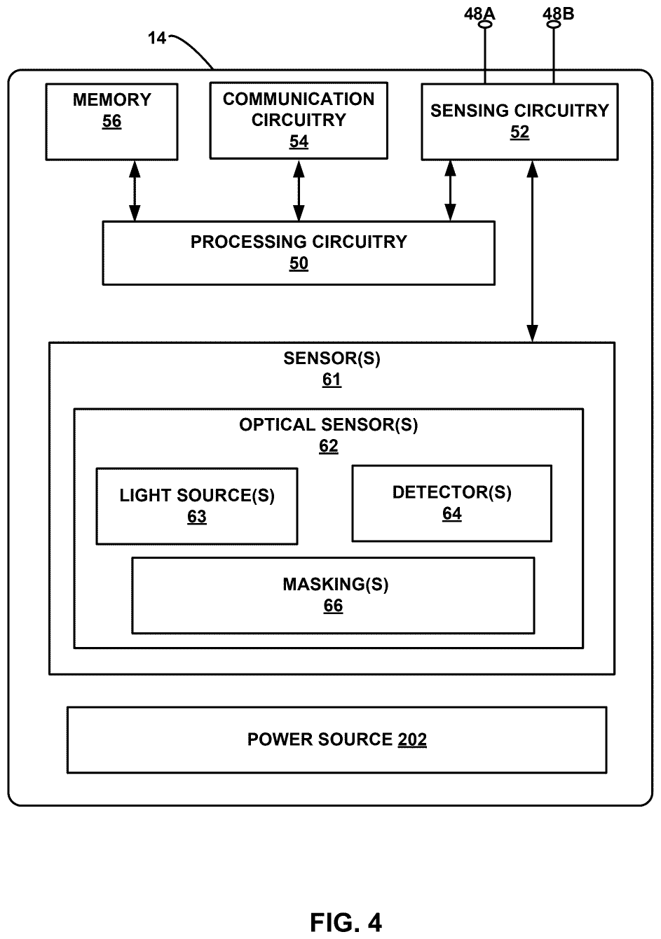

FIG. 2 is a functional schematic diagram of IMD 14, and FIG. 3 is a conceptual perspective schematic diagram of IMD 14, according to various examples described in this disclosure. IMD 14 may be a leadless, subcutaneously implantable monitoring device including proximal electrode 48B located at proximal end 222, distal electrode 48A located at distal end 220 (collectively “electrodes 48”), optical sensor(s) 62, integrated antenna 226, electrical circuitry 200 and power source 202. In particular, electrical circuitry 200 is coupled to proximal electrode 48B and distal electrode 48A to sense cardiac signals and monitor events. Electrical circuitry 200 may also connected to transmit and receive communications via integrated antenna 226. Power source 202 provides power to electrical circuitry 200, as well as to any other components that require power. Power source 202 may include one or more energy storage devices, such as one or more rechargeable or non-rechargeable batteries. In some examples, electrical circuitry 200 includes processing circuitry 50 and a storage device, such as memory 56, as shown in FIG. 4, the memory 56 being operatively coupled to the processing circuitry 50 and configured to store data and/or instructions.

In the example shown in FIG. 2, electrical circuitry 200 may receive raw EGM or EMG (electromyography) signals monitored by proximal electrode 48B and distal electrode 48A and raw optical signals monitored by optical sensor(s) 62. Electrical circuitry 200 may include components/modules for converting the raw EGM signal to a processed EGM signal that can be analyzed to detect sense events and for converting the raw optical signals to calibrated processed optical signal(s) that can be analyzed to detect sense events. Although not shown, electrical circuitry 200 may include any discrete and/or integrated electronic circuit components that implement analog and/or digital circuits capable of producing the functions described for analyzing optical signal(s) to determine a health condition status of a patient. For example, the electrical circuitry 200 may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, a processor (shared, dedicated, or group) that executes one or more software or firmware programs, memory devices, or any other suitable components or combination thereof that provide the described functionality.

In one example, electrical circuitry 200 includes a sensing unit for monitoring the EGM signal detected by the respective proximal and distal electrodes 48A and 48B and light signals received by the optical sensor(s) 62, respectively. In one example, electrical circuitry 200 includes processing circuitry 50 that is utilized to receive information regarding sensed events and implements one or more algorithms for determining a health condition status of a patient. In addition, the analog voltage signals received from electrodes 48A and 48B may be passed to analog-to-digital (A/D) converters included in the electrical circuitry 200 and stored in a memory unit (not shown) included as part of electrical circuitry 200 for subsequent analysis with firmware executed by the processor included as part of electrical circuitry 200.

In the examples shown in FIGS. 2-3, IMD 14 may include container 15 and an insulative cover 76. In some examples, insulative cover 76 may include optical window 75. In some examples, optical window 75 may be formed of the same material as insulative cover 76. In some examples, optical window 75 may be a portion of insulative cover 76. Electrode 48A and electrode 48B may be formed or placed on an outer surface of cover 76. Electrical circuitry 200 may be formed or placed on an inner surface of cover 76, or within container 15. In some examples, antenna 226 is formed or placed on the inner surface of cover 76. In other examples, antenna 226 is formed or placed on the outer surface of cover 76, and in other examples, antenna 226 may be formed or placed at least partially on the inner surface and partially on the outer surface of cover 76. In some examples, insulative cover 76 may be positioned over an open container 15 such that container 15 and cover 76 form housing 20 and enclose electrical circuitry 200 (and in some cases antenna 226) and protect the circuitries from fluids such as body fluids. For example, housing 20 may be a hermetically-sealed housing configured for subcutaneous implantation within the patient, wherein at least the power source 202, memory, and processing circuitry 50 are within the hermetically-sealed case, and in some examples, optical sensor(s) 62 are within the hermetically-sealed case.

Electrical circuitry 200 may be formed on the inner side of insulative cover 76, such as by using flip-chip or wire bond integrated circuit packaging technology. Insulative cover 76 may be flipped onto a container 15. When flipped and placed onto container 15, the components of IMD 14 formed on the inner side of insulative cover 76 may be positioned in a gap defined by container 15. Electrodes 48 and antenna 226 (when placed or formed on the outer surface of cover 76) may be electrically connected to sensing circuitry 52 and communication circuitry 54 (FIG. 4), respectively, e.g., through one or more vias formed through insulative cover 76. Insulative cover 76 may be formed of sapphire (i.e., corundum), glass, and/or any other suitable insulating material. Container 15 may be formed from any suitable material configured to house electrical circuitry 200, support and mate with cover 76 to isolate electrical circuitry 200 from contact with tissue and/or fluids of patient 12, and to be implantable within patient 12. In some examples, container 15 may house power source 202 (e.g., a battery). In some examples, container 15 may also be electrically conductive. For example, container 15 may be formed from titanium or any other suitable material (e.g., a biocompatible material). Electrodes 48 may be formed from any of stainless steel, titanium, platinum, iridium, or alloys thereof. In addition, electrodes 48 may be coated with a material such as titanium nitride or fractal titanium nitride, although other suitable materials and coatings for such electrodes may be used.

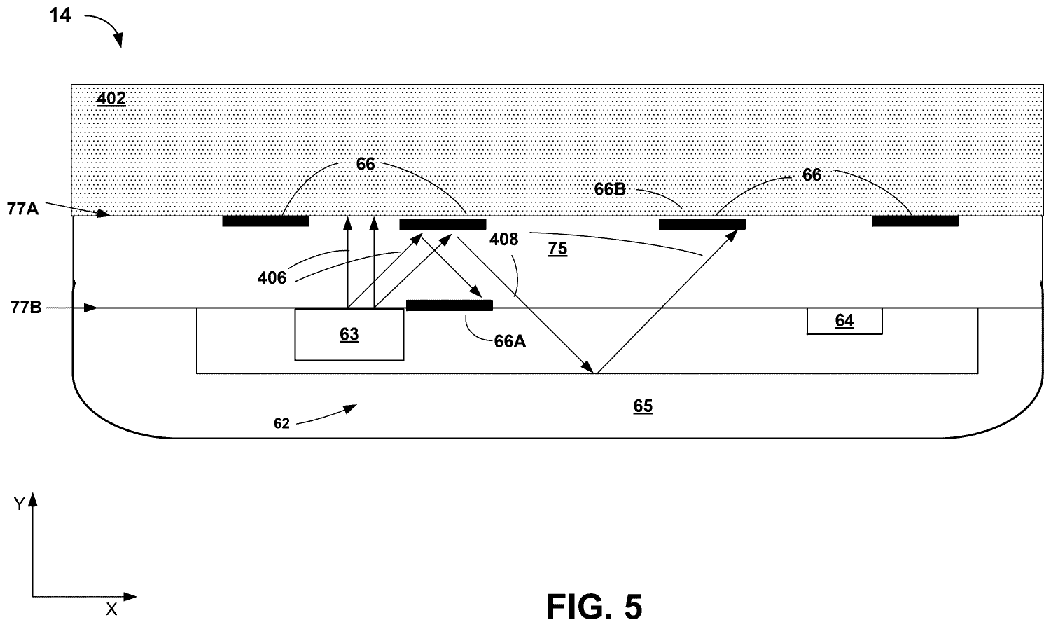

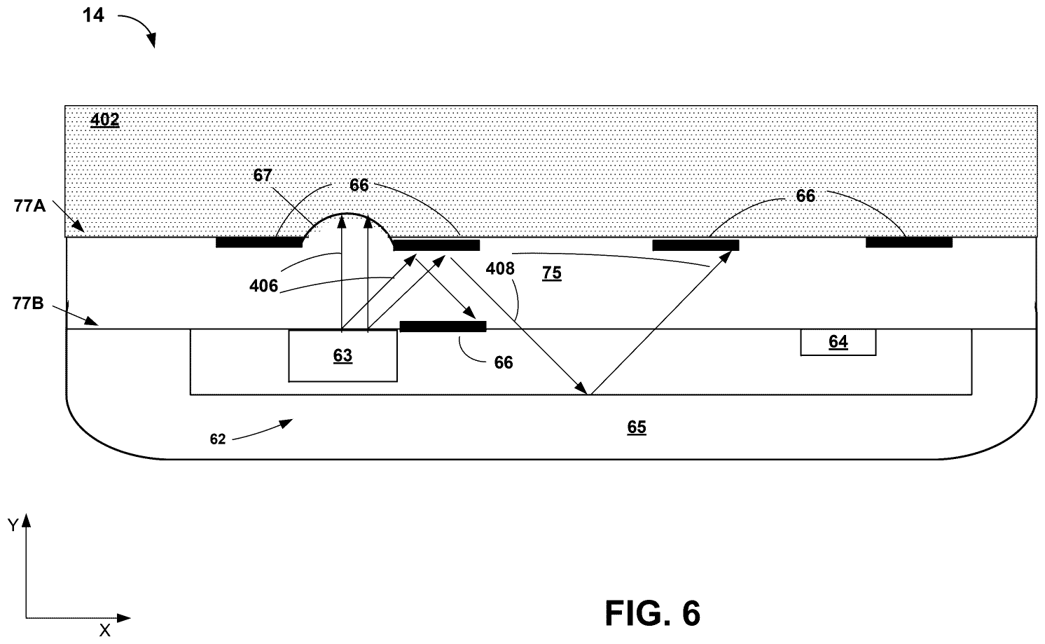

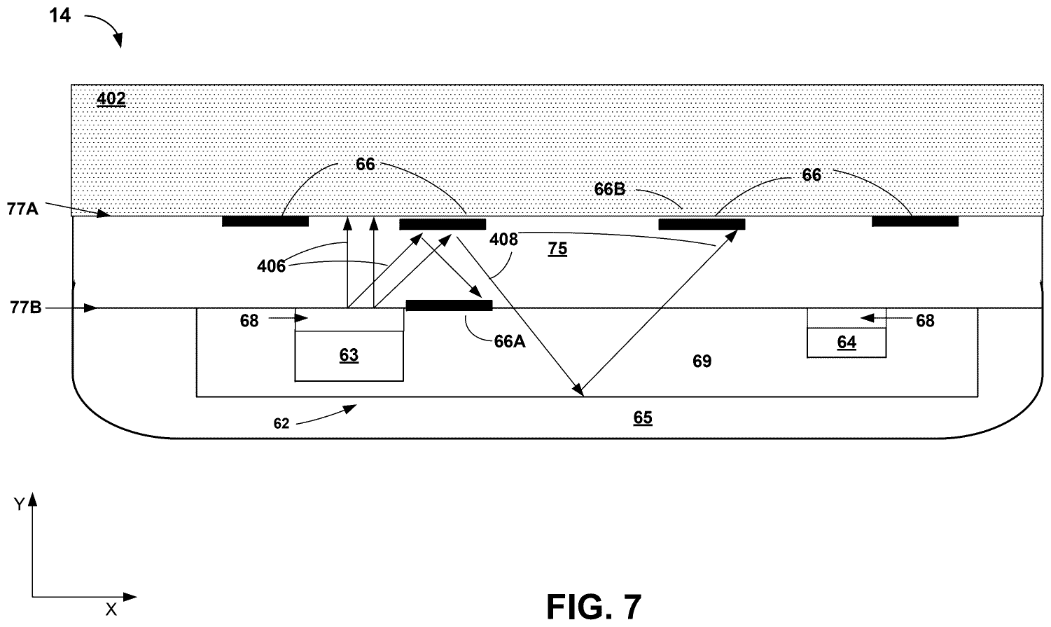

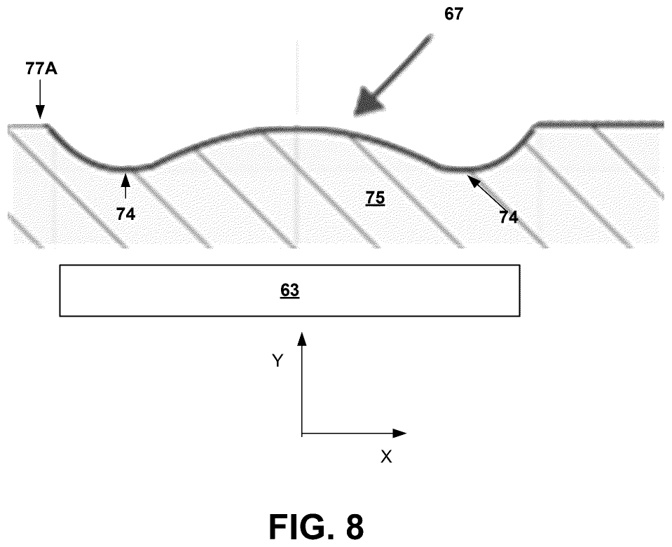

FIG. 4 is a block diagram illustrating an example of IMD 14 of FIGS. 1-3. FIG. 4 is described with reference to FIGS. 5-8. FIG. 5 is a conceptual cross-sectional side-view of an example IMD 14 including masking 66 in a first configuration, FIG. 6 is a conceptual cross-sectional side-view of the example IMD 14 including masking 66 and an integrated lens 67 in a second configuration, FIG. 7 is a conceptual cross-sectional side-view of the example IMD 14 including the masking 66 and underfill 68 in a third configuration, and FIG. 8 is a conceptual cross-sectional side-view of an example of an integrated lens 67.

As shown in FIG. 4, IMD 14 includes processing circuitry 50, memory 56, one or more sensor(s) 61, which may include one or more optical sensor(s) 62, sensing circuitry 52 coupled to sensors 61 and electrodes 48 (e.g., electrodes 48A and 48B), power source 202, and communication circuitry 54. Power source 202 provides operational power for processing circuitry 50, sensing circuitry 52, sensor(s) 61, communication circuitry 54, and memory 56. As used herein, “sensors” may refer to any sensors described herein, including electrodes 48 and optical sensor 62.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be coupled to electrodes 48 to sense electrical signals of the heart of patient 12, for example by selecting electrodes 48 and polarity, used to sense an ECG as controlled by processing circuitry 50. Sensing circuitry 52 may sense the ECG from electrodes 48 in order to facilitate monitoring the electrical activity of the heart. In some examples, electrodes 48 may be configured to sense other electrogram signals of patient 12, and/or impedance of tissue fluid proximate the electrodes 48. Sensing circuitry 52 also may monitor signals from sensors 61, which may include one or more accelerometers (e.g., 3-axis accelerometers), pressure sensors, temperature sensors, heart sound sensors (e.g., microphones or accelerometers), optical sensors 62, or other sensors.

In some examples, sensing circuitry 52 may include one or more filters and amplifiers for filtering and amplifying signals received from electrodes 48 and/or sensors 61. In some examples, sensing circuitry 52 may sense or detect physiological parameters, such as heart rate, blood pressure, respiration, and other physiological parameters associated with a patient. Sensing circuitry 52 and processing circuitry 50 may store ECG data and/or other physiological parameter data in memory 56.

Communication circuitry 54 may include any suitable hardware, firmware, software or any combination thereof for communicating with another device, such as external device 24 (FIG. 1), another networked computing device, or another IMD or sensor. Under the control of processing circuitry 50, communication circuitry 54 may receive downlink telemetry from, as well as send uplink telemetry to external device 24 or another device with the aid of an internal or external antenna, e.g., antenna 226. In addition, processing circuitry 50 may communicate with a networked computing device via external device 24 and a computer network, such as the Medtronic CareLink® Network. Antenna 226 and communication circuitry 54 may be configured to transmit and/or receive signals via inductive coupling, electromagnetic coupling, Near Field Communication (NFC), Radio Frequency (RF) communication, Bluetooth®, Wi-Fi, or other proprietary or non-proprietary wireless communication schemes. Communication antenna 226 may telemeter data at a high frequency, such as around 2.4 gigahertz (GHz).

In some examples, memory 56 includes computer-readable instructions that, when executed by processing circuitry 50, cause IMD 14 and processing circuitry 50 to perform various functions attributed to IMD 14 and processing circuitry 50 herein. Memory 56 may include any volatile, non-volatile, magnetic, optical, or electrical media, such as a random-access memory (RAM), dynamic RAM (DRAM), static RAM (SRAM), read-only memory (ROM), non-volatile RAM (NVRAM), electrically erasable programmable ROM (EEPROM), flash memory, or any other digital media. Memory 56 may store, as examples, programmed values for one or more operational parameters of IMD 14 and/or data collected by IMD 14, e.g., posture, heart rate, activity level, respiration rate, oxygen saturation, blood pressure, and other parameters, as well as digitized versions of physiological signals, e.g., optical signals or ECG signals, sensed by IMD 14, for transmission to another device using communication circuitry 54. In some examples, memory 56 is used to store program instructions for execution by processing circuitry 50. Memory 56 may be used by software or applications running on processing circuitry 50 and/or sensing circuitry 52 to temporarily store information during program execution.

In the illustrated example, IMD 14 includes processing circuitry 50 and an associated memory 56, sensing circuitry 52, one or more sensors 61, and the communication circuitry 54 coupled to antenna 226 as described above. However, IMD 14 need not include all of these components, or may include additional components.

Sensing circuitry 52 may additionally be coupled to optical sensor 62 to sense a physiological characteristic, signal, or parameter, of patient 12, e.g., a blood pressure, an oxygenation of arterial blood, perfusion and oxygenation of tissue, a heart rate, a respiration rate, a movement, or any suitable physiological characteristic. Optical sensor 62 may include one or more detector(s) 64 configured to receive and/or detect light, such light originating from one or more light source(s) 63 and reflected and/or scattered from tissue 402 (FIGS. 5-7). Light received and/or detected by detector 64 may be referred to as optical signals. In some examples, optical sensor 62 may be configured to receive light reflected by blood in one or more blood vessels. In some examples, light source 63 may be configured to emit light signals belonging to a particular wavelength spectrum. Some examples of a particular wavelength spectrum may be an amber wavelength spectrum, a green wavelength spectrum, yellow wavelength spectrum, blue wavelength spectrum, red wavelength spectrum, an infrared wavelength spectrum, or any other suitable wavelength spectrum. Detector 64 may be configured to receive optical signals of the corresponding particular wavelength spectrum, e.g., facilitated by filtering and/or or detector material selection.

Optical sensor 62 may also include one or more masking(s) 66. In some examples, blocking light comprises reflecting the light, absorbing the light, or attenuating some or all of the light. In some examples, it may be preferable to absorb the light intended to be blocked, rather than to reflect the light, to prevent that light from being backscattered by tissue 402 towards detector 64. In some examples, masking 66 may also be configured, such as being coated with a particular coating, to spectrally filter light, e.g., to block some wavelengths of light more than others. In some examples, masking 66 may comprise other spectral filters, e.g., infrared filter(s), visible filter(s), ultraviolet filter(s), or any number of filters configured to absorb any suitable spectral band.

Sensing circuitry 52 and/or processing circuitry 50 may be configured to control operation of optical sensor 62, e.g., to determine parameters of light source 63 such as brightness level and/or pulsing emission of the light, and parameters of detector 64 such as gain. In some examples, optical sensor 62 may be a photoplethysmography (PPG) sensor, an oxygenation sensor, a blood pressure sensor, a heart rate sensor, or any suitable optical sensor configured to detect a physiological characteristic and/or parameter of patient 12.

In the examples shown in FIGS. 5-7, IMD 14 is implanted within patient 12 and is in contact with tissue 402. Tissue 402 includes blood vessels, such as arterioles having relatively high dynamic optical absorbance through the cardiac cycle, and tissue structures that do not have relatively high dynamic optical absorbance through the cardiac cycle.

In some examples, optical window 75 may comprise sapphire to transmit light from light source 63 into tissue 402 and/or to detect light within tissue 402 at detector 64. In some examples, optical window 75 may comprise a single crystal sapphire or glass. Light transmission into and/or out of IMD 14 through optical window 75 may result in reflection and refraction at interfaces with optical window 75, such as due to index of refraction differences between a high refractory index optical window 75, such as sapphire, and low refractory index tissue 402 and/or low refractory index gas within IMD 14. In some examples, reflection and/or refraction at optical window 75 interfaces may result in attenuation of emitted light 406 from light source 63 into tissue 402, reducing the overall amount of light that is to be detected by detector 64 of IMD 14.

In some examples light transmission from light source 63 into optical window 75 may result in some emitted light 406 from light source 63 being reflected light 408. In some examples, emitted light 406 may reflect 408 between a top side 77A and a bottom side 77B within optical window 75, between a top side 77A optical window 75 and device case 65, and/or between a bottom side 7B of optical window 75 and device case 65. In some examples, emitted light reflecting may result in light emitted from light source 63 being transmitted to detector 64 without the reflected light exiting IMD 14, which may result in interference with measurements of light emitted by light source 63 through optical window 75 to tissue 402 and received from tissue 402 by detector 64. In some examples, ambient light (e.g., light from a different light source external to optical sensor 62) from within the ambient environment may enter tissue 402 and be detected by detector 64. In some examples, ambient light may interfere with measurements of light emitted by light source 63 through optical window 75 to tissue 402 and received from tissue 402 by detector 64.

In some examples, IMD 14 includes a masking 66 positioned at one or more portions of the optical window 75. In some examples, masking 66 may be configured to absorb a portion of the emitted light, such as the emitted light internally reflected within optical window 75. In some examples, masking 66 may comprise absorptive masking. In some examples, masking 66 may be coated with an absorptive material to absorb reflected light so light received by detector 64 is light that is attenuated by tissue 402. In some examples, masking 66 may reduce internal reflections within optical window 75, may reduce ambient light transmission and interference within IMD 14. In some examples, masking 66 may be particularly positioned to reduce clipping or limiting light transmission based on critical angles of reflection. In some examples, the X-direction, as shown in FIGS. 5-7, may be referred to as a planar direction, and the Y-direction, as shown in FIGS. 5-7, may be referred to as a vertical direction. In some examples, masking 66 may be positioned adjacent to light source 63 in the planar direction.

Processing circuitry of IMD 14 or external device 24 may be configured to determine a physiological characteristic (e.g., a physiological signal or parameter) of patient 12 based on optical sensing light (e.g., light that is emitted by a light source 63 of optical sensor 62 and passes into tissue 402 and interacts with tissue 402, such as through absorption and scattering) detected by one or more detector(s) 64 of optical sensor 62. In some examples, reflected light 408 (e.g., light that is from one or more of light source 63 of optical sensor 62 that is reflected internally within IMD 14) and/or ambient light (e.g., light from a different light source external to optical sensor 62) from within tissue 402 may interfere with measurements of light detected by optical sensor 62. In some examples, reflected light 408 and/or ambient light may contribute to noise or reduce the effective dynamic range of detector 64.

In some examples, light source 63 may comprise a plurality of light sources. In other examples, light source 63 may comprise a single light source, e.g., an LED. In some examples, light source 63 may be configured to emit light within a substantially large solid angle, e.g., a Lambertian or substantially Lambertian light source. In other examples, light source 63 may be configured to emit light within particular range of angles and in a particular angular direction, e.g., a conical solid angle having an apex angle between 20 degrees and 180 degrees and directed at any suitable angle, e.g., from zero degrees (substantially normal, or perpendicular, to the outer surface of insulative cover 76) to 90 degrees (substantially parallel to the outer surface of insulative cover 76, e.g., a glancing angle along the outer surface of insulative cover 76). In some examples, light source 63 may include, or be used in conjunction with, beam forming and/or shaping optics, such as lenses, diffraction gratings, holographic optical elements, or the like. In some examples, light source 63 may include a coherent light source, e.g., temporally and/or spatially coherent, such as a laser, a VCSEL, or the like, which may have a divergence angle of less than or equal to 20 degrees.

In some examples, detector 64 may comprise a single detecting element, e.g., a relatively large area photodiode. In some examples, detector 64 may comprise an array in one or two dimensions of a plurality of detectors. In some examples, detector 64 has an area that is smaller than the area of optical window 75. In some examples, detector 64 may have an area that covers substantially all, or a large portion of the area of optical window 75.

In some examples, masking 66 may be directly mounted on optical window 75. In some examples, optical sensor 62 may include one or more lenses positioned on light source 63. In some examples, one or more lenses positioned on light source 63 may collimate light to reduce a high angle reflection on optical window 75.

In some examples, optical window 75 includes top side 77A and bottom side 77B, the top side 77A being positioned adjacent to tissue 402 when IMD 14 is implanted and bottom side 77B being positioned to optical sensor 62. In some examples, top side 77A is on an opposite side of optical window 75 compared to bottom side 77B. In some examples, masking 66 may include a masking 66A positioned on a portion of bottom side 77B and a masking 66B positioned on a portion of top side 77A. In some examples, masking 66A on bottom side 77B may be positioned adjacent to light source 63 in the planar direction and positioned between light 63 and detector 64 in the planar direction. In some examples, masking 66B positioned on a top side 77A may be positioned between masking 66A and detector 64 in the planar direction. In some examples, masking 66A may extend, in the planar direction, between light source 63 and detector 64. In some examples, masking 66A may extend, in the planar direction, completely between light source 63 and detector 64.

In some examples, masking 66, such as masking 66A, is configured to absorb light, such as reflected light 408 that is reflected internally within IMD 14, to reduce the amount of reflected 408 detected by detector 64. In some examples, masking 66, such as masking 66B, is configured to absorb light, such as ambient light in tissue 402, to reduce the amount of ambient light detected by detector 64.

In some examples, as shown in FIG. 6, optical window 75 may include an integrated lens 67 and light source 63 may be positioned to emit light to integrated lens 67. In some examples, integrated lens 67 may include a convex shape and light source 63 may be positioned to emit light to the convex shape of the integrated lens 67. FIG. 8 shows an example of integrated lens 67. In some examples, as shown in FIG. 8, integrated lens 67 may include at least one portion 74 of integrated lens 67 positioned below (e.g. closer to light source 63) top side 77A in the vertical direction. In some examples, integrated lens 67 positioned directly above light source 63 in the vertical direction (e.g., y-direction). In some examples, optical window 75 including integrated lens 67 may reduce internal reflection of light emitted by light source 63. In some examples, optical window 75 may additionally or alternatively include an integrated lens positioned directly above detector 64 in the vertical direction (e.g., y-direction).

In some examples, a distance between light source 63 and detector 64 may determine a depth the emitted light reaches tissue 402. For example, a greater a distance between light source 63 and detector 64 the greater the depth from optical window 75 the emitted light reaches tissue 402 and is then received by detector 64. In some examples, light sources 63 may include a first light source and a second light source, detectors 64 may include a first detector and a second detector. In some examples, a distance between the first light source and the first detector may be different than a distance between the second light source and the second detector. In some examples, optical sensor 62 having light sources 63 and detectors 64 being separated by different distances, optical sensor 62 is able to sense tissue 402 at different tissue depths which increases data received by detectors 64 from tissue 402. In some examples, optical sensor 62 may have light sources 63 and detectors 64 placed in an array of locations throughout an area of optical window 75 such that optical sensor 62 is able to sense a plurality of tissue volumes contacting IMD 14 (optical vectors). In some examples, optical sensor 62 may selectively sample discrete tissue volumes and measure their optical properties based on the array of optical vectors. In some examples, optical sensor 62 may be able to select one or more optical vectors to use as inputs based on optical properties for each optical vector. In some examples, when external device 24 includes one or more light sources, a number of optical vectors may increase above a threshold to allow additional discrete tissue volumes to be tested to optimize signal to noise ratio and/or a particular physiological signal of interest.

In some examples, detector 64 may be positioned at a particular distance from light source 63 to reduce optical harmonics of reflected light 408 that may be received by detector 64.

In some examples, as shown in FIG. 7, IMD 14 comprises an underfill 68 positioned between one or more of light source 63 or detector 64 and optical window 75. In some examples, an index of refraction of the underfill may be within 20% of an index of refraction of the optical window. In some examples, material of the underfill 68 index matches a material of optical window 75. For example, when optical window 75 comprises sapphire, underfill 68 comprises material with an index of refraction similar to the index of refraction of sapphire. For example, underfill 68 may have an index of refraction between 1.7 and 1.9. In some examples, underfill 68 may have an index of refraction between 1.4 and 2.0. In some examples, underfill 68 may have an index of refraction between 1.45 and 1.77. In some examples, IMD 14 having underfill 68 being positioned between light source 63 and optical window 75 may reduce an amount of light that is refracted at interfaces of optical window 75, such as interface at bottom side 77B of optical window. In some examples, IMD 14 having underfill 68 being positioned between light source 63 and optical window 75 may reduce an amount of emitted light 406 is reflected, which improves efficiency of emitted light 406 being transmitted to tissue 402 and/or reduces internal device reflections into the photodiode. In some examples, an area between device case 65 and optical window 75 may include absorptive backfill 69 configured to absorb reflected light 408 positioned in at least some of the area between device case 65 and optical window 75. In some examples, the absorptive backfill may be a gas. In some examples, the absorptive backfill may be a fluid or a solid. In some examples, absorptive backfill may comprise a highly absorptive material on a side of the absorptive backfill facing light source 63 and/or detector 64.

In some examples, optical window 75 comprises a plurality of vias. In some examples, optical window 75 comprises a plurality of vias that are positioned in optical window 75 between light source 63 and detector 64 in the planar direction. In some examples, placement of vias between light source 63 and detector 64 may attenuate light transmitted in optical window 75 parallel to the plane of the window to provide further masking control to minimize light transmission, such as with no patient physiological signal.

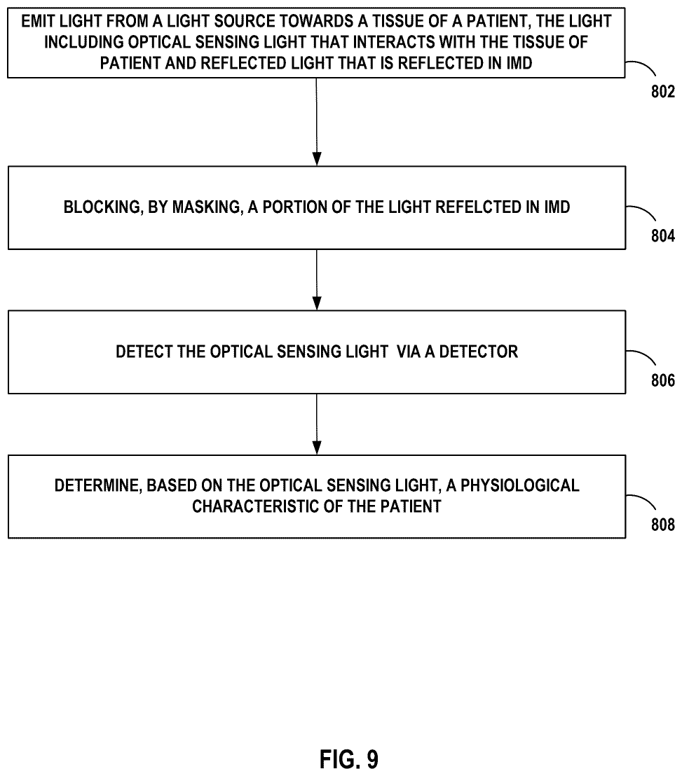

FIG. 9 is a flow diagram of an example method of determining a physiological characteristic of a patient, according to various examples described in this disclosure. Although the example technique of FIG. 1 is described with respect to medical systems 10, IMD 14, and masking 66 of FIGS. 1-8, the example technique of FIG. 9 may be performed using any system including an implantable medical device and masking described herein.

Light source 63 may emit light 406 towards a tissue 402 of patient 12 (802). For example, sensing circuitry 52 and/or processing circuitry 50 may cause light source 63 to emit broadband light or light having a specific wavelength band, e.g., IR light. The light may include optical sensing light (e.g., light that is emitted by a light source 63 of optical sensor 62 and passes into tissue 402 and interacts with tissue 402, such as through absorption and scattering) that interacts with tissue 402 of patient 12 and reflected light 408 that is reflected in IMD 14. In some examples, light source 63 may emit light through underfill 68, the underfill 68 to the optical window. In some examples, the underfill 68 is positioned directly between the light source 63 and the optical window 75.

Masking 66 positioned on a portion of optical window 75 may block at least a portion of the reflected light 408 (804). Detector 64 may detect the optical sensing light (e.g., light that is emitted by a light source 63 of optical sensor 62 and passes into tissue 402 and interacts with tissue 402, such as through absorption and scattering) (806). Sensing circuitry 52 and/or processing circuitry 50 may receive a signal indicative of the detected light from detector 64. Sensing circuitry 52 and/or processing circuitry 50 may determine a physiological characteristic of patient 12 based, at least, on the detected optical sensing light of the light (808).

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media. Various aspects of the techniques may enable the following examples.
Example 1: An implantable medical device includes a housing comprising an optical window; an optical sensor within the housing and configured to emit light to the optical window; and a masking on a portion of the optical window, the masking configured to absorb a portion of the emitted light internally reflected within the optical window.
Example 2: The implantable medical device of example 1, wherein the optical window comprises an integrated lens, and the optical sensor comprises a light source positioned to emit light to the integrated lens.
Example 3: The implantable medical device of example 2, wherein the integrated lens includes a convex shape, and the light source is positioned to emit light to the convex shape of the integrated lens.
Example 4: The implantable medical device of example 1, wherein the optical sensor comprises a light source positioned to emit light, and the masking is positioned adjacent to the light source.
Example 5: The implantable medical device of example 1, wherein the optical window includes a first side and a second side, the first side being positioned adjacent to the optical sensor and the second side, opposite of the first side, to be positioned adjacent to tissue when the implantable medical device is implanted, wherein the masking includes a first masking on a portion of the first side of the optical window and second masking on a portion of the second side of the optical window.
Example 6: The implantable medical device of example 5, wherein the optical sensor comprises a light source configured to emit light and a detector configured to detect light, and the first masking is positioned adjacent to the light source and between the light source and the detector.
Example 7: The implantable medical device of example 6, wherein the second masking is positioned between the first masking and the detector.
Example 8: The implantable medical device of any of examples 1-7, wherein the optical sensor comprises a light source configured to emit light and a detector configured to detect light, and the implantable medical device further comprises underfill positioned directly between at least one of the light source or the detector and the optical window.
Example 9: The implantable medical device of example 8, wherein an index of refraction of the underfill is within 20% of an index of refraction of the optical window.
Example 10: The implantable medical device of any of examples 1-9, wherein the optical sensor comprises a light source configured to emit light and a detector configured to detect light, and the optical window comprises a plurality of vias positioned in the optical window between the light source and the detector.
Example 11: The implantable medical device of any of examples 1-10, wherein the housing further comprises a device case, and the implantable medical device further comprises absorptive backfill positioned between the optical window and the device case.
Example 12: The implantable medical device of example 11, wherein the absorptive backfill comprises a gas or a fluid.
Example 13: A method of determining a physiological characteristic of a patient includes emitting light, by a light source of an optical sensor positioned in an implantable medical device (IMD), to an optical window of the IMD and towards a tissue of the patient, the light including optical sensing light that is to interact with the tissue of the patient and reflected light that is to be reflected in the IMD; blocking, by masking positioned on a portion of the optical window, at least a portion of the reflected light in the IMD; detecting, by a detector, the optical sensing light; and determining, by processing circuitry and based, at least, on the detected optical sensing light, the physiological characteristic of the patient.
Example 14: The method of example 13, wherein the optical window comprises an integrated lens and the light source is positioned to emit light to the integrated lens.
Example 15: The method of example 14, wherein the integrated lens includes a convex shape, and the light source is positioned to emit light to the convex shape of the integrated lens.
Example 16: The method of example 13, wherein the optical window includes a first side and a second side, the first side being positioned adjacent to the optical sensor and the second side, opposite of the first side, to be positioned adjacent to tissue when the IMD is implanted in the patient, wherein the masking includes first masking on a portion of the first side of the optical window and second masking on a portion of the second side of the optical window.
Example 17: The method of example 16, wherein the optical sensor further includes a detector to detect light, and the first masking is positioned adjacent to the light source and between the light source and the detector.
Example 18: The method of example 17, wherein the second masking is positioned between the first masking and the detector.
Example 19: The method of example 13, wherein the optical sensor further includes a detector to detect light, and the method further comprises: emitting light, by the light source, through underfill, positioned directly between the light source and the optical window, to the optical window.
Example 20: The method of example 19, wherein an index of refraction of the underfill is within 20% of an index of refraction of the optical window.

Various examples have been described. These and other examples are within the scope of the following claims.

What is claimed is:

1. An implantable medical device comprising:
a housing comprising an optical window;
an optical sensor within the housing and configured to emit light to the optical window; and
a masking on a portion of the optical window, the masking configured to absorb a portion of the emitted light internally reflected within the optical window.

2. The implantable medical device of claim 1, wherein the optical window comprises an integrated lens, and
the optical sensor comprises a light source positioned to emit light to the integrated lens.

3. The implantable medical device of claim 2, wherein the integrated lens includes a convex shape, and the light source is positioned to emit light to the convex shape of the integrated lens.

4. The implantable medical device of claim 1, wherein the optical sensor comprises a light source positioned to emit light, and
the masking is positioned adjacent to the light source.

5. The implantable medical device of claim 1, wherein the optical window includes a first side and a second side, the first side being positioned adjacent to the optical sensor and the second side, opposite of the first side, to be positioned adjacent to tissue when the implantable medical device is implanted,
wherein the masking includes a first masking on a portion of the first side of the optical window and second masking on a portion of the second side of the optical window.

6. The implantable medical device of claim 5, wherein the optical sensor comprises a light source configured to emit light and a detector configured to detect light, and
the first masking is positioned adjacent to the light source and between the light source and the detector.

7. The implantable medical device of claim 6, wherein the second masking is positioned between the first masking and the detector.

8. The implantable medical device of claim 1, wherein the optical sensor comprises a light source configured to emit light and a detector configured to detect light, and the implantable medical device further comprises underfill positioned directly between at least one of the light source or the detector and the optical window.

9. The implantable medical device of claim 8, wherein an index of refraction of the underfill is within 20% of an index of refraction of the optical window.

10. The implantable medical device of claim 1, wherein the optical sensor comprises a light source configured to emit light and a detector configured to detect light, and
the optical window comprises a plurality of vias positioned in the optical window between the light source and the detector.

11. The implantable medical device of claim 1, wherein the housing further comprises a device case, and
the implantable medical device further comprises absorptive backfill positioned between the optical window and the device case.

12. The implantable medical device of claim 11, wherein the absorptive backfill comprises a gas or a fluid.

13. A method of determining a physiological characteristic of a patient, the method comprising:
emitting light, by a light source of an optical sensor positioned in an implantable medical device (IMD), to an optical window of the IMD and towards a tissue of the patient, the light including optical sensing light that is to interact with the tissue of the patient and reflected light that is to be reflected in the IMD;
blocking, by masking positioned on a portion of the optical window, at least a portion of the reflected light in the IMD;
detecting, by a detector, the optical sensing light; and
determining, by processing circuitry and based, at least, on the detected optical sensing light, the physiological characteristic of the patient.

14. The method of claim 13, wherein the optical window comprises an integrated lens and the light source is positioned to emit light to the integrated lens.

15. The method of claim 14, wherein the integrated lens includes a convex shape, and the light source is positioned to emit light to the convex shape of the integrated lens.

16. The method of claim 13, wherein the optical window includes a first side and a second side, the first side being positioned adjacent to the optical sensor and the second side, opposite of the first side, to be positioned adjacent to tissue when the IMD is implanted in the patient,
wherein the masking includes first masking on a portion of the first side of the optical window and second masking on a portion of the second side of the optical window.

17. The method of claim 16, wherein the optical sensor further includes a detector to detect light, and
the first masking is positioned adjacent to the light source and between the light source and the detector.

18. The method of claim 17, wherein the second masking is positioned between the first masking and the detector.

19. The method of claim 13, wherein the optical sensor further includes a detector to detect light, and the method further comprises:
emitting light, by the light source, through underfill, positioned directly between the light source and the optical window, to the optical window.

20. The method of claim 19, wherein an index of refraction of the underfill is within 20% of an index of refraction of the optical window.
